(19) United States
(12) Patent Application Publication
(10) Pub. No.: US 2007/0280936 A1
(43) Pub. Date: Dec. 6, 2007

(54) METHOD FOR TREATING PSORIASIS

(75) Inventors: Emma E. Moore, Seattle, WA (US); Kevin P. Foley, Cambridge, MA (US); Karen L. Madden, Bellevue, WA (US); Yue Yao, Kenmore, WA (US); Scott R. Presnell, Tacoma, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(73) Assignee: ZymoGenetics, Inc.

(21) Appl. No.: 11/753,498

(22) Filed: May 24, 2007

Related U.S. Application Data
(63) Continuation of application No. 10/466,823, filed on Jan. 29, 2004, now abandoned.
(60) Provisional application No. 60/264,219, filed on Jan. 25, 2001.

(51) Int. Cl. A61K 39/395; A61P 17/06

(52) U.S. Cl. 424/135.1; 424/130.1; 424/139.1; 514/789

(57) ABSTRACT

A method for treating psoriasis. An antagonist to IL-17Dβ9 (IL-17D) is administered to treat psoriasis. The antagonist can be an antibody that binds to IL-17Dβ9 or its receptor or a soluble receptor that binds to IL-17Dβ9 or a membrane-spanning protein-5 (MSP-5).

This application is a continuation of U.S. application Ser. No. 10/466,823, filed Jan. 24, 2002, which is the National Stage of International Application No. PCT/US02/02244, filed Jan. 24, 2002, which claims the benefit of U.S. Provisional Application Ser. No. 60/264,219, filed Jan. 25, 2001, all of which are herein incorporated by reference.

### BACKGROUND OF THE INVENTION

The teachings of all of the references cited herein are incorporated in their entirety herein by reference.

Psoriasis is one of the most common dermatologic diseases, affecting up to 1 to 2 percent of the world's population. It is a chronic inflammatory skin disorder characterized by erythematous, sharply demarcated papules and rounded plaques, covered by silvery micaceous scale. The skin lesions of psoriasis are variably pruritic. Traumatized areas often develop lesions of psoriasis. Additionally, other external factors may exacerbate psoriasis including infections, stress, and medications, e.g. lithium, beta blockers, and anti-malarials.

The most common variety of psoriasis is called plaque type. Patients with plaque-type psoriasis will have stable, slowly growing plaques, which remain basically unchanged for long periods of time. The most common areas for plaque psoriasis to occur are the elbows knees, gluteal cleft, and the scalp. Involvement tends to be symmetrical. Inverse psoriasis affects the intertriginous regions including the axilla, groin, submammary region, and navel, and it also tends to affect the scalp, palms, and soles. The individual lesions are sharply demarcated plaques but may be moist due to their location. Plaque-type psoriasis generally develops slowly and runs an indolent course. It rarely spontaneously remits.

Eruptive psoriasis (guttate psoriasis) is most common in children and young adults. It develops acutely in individuals without psoriasis or in those with chronic plaque psoriasis. Patients present with many small erythematous, scaling papules, frequently after upper respiratory tract infection with beta-hemolytic streptococci. Patients with psoriasis may also develop pustular lesions. These may be localized to the palms and soles or may be generalized and associated with fever, malaise, diarrhea, and arthralgias.

About half of all patients with psoriasis have fingernail involvement, appearing as punctate pitting, nail thickening or subungual hyperkeratosis. About 5 to 10 percent of patients with psoriasis have associated joint complaints, and these are most often found in patients with fingernail involvement. Although some have the coincident occurrence of classic Although some have the coincident occurrence of classic rheumatoid arthritis, many have joint disease (psoriatic arthritis) that falls into one of five type associated with psoriasis: (1) disease limited to a single or a few small joints (70 percent of cases); (2) a seronegative rheumatoid arthritis-like disease; (3) involvement of the distal interphalangeal joints; (4) severe destructive arthritis with the development of “arthritis mutilans”; and (5) disease limited to the spine.

A number of treatments exist for psoriasis but they do not result in satisfactory remission of the disease. Thus there is a need to discover new therapies that more effectively treat the disease.

### DESCRIPTION OF THE INVENTION

The present invention fills this need by providing for a method for treating psoriasis or psoriatic arthritis, which comprises administering to a mammal afflicted with psoriasis or psoriatic arthritis an antagonist to interleukin-17 (also known as Ztgfβ-9). The antagonist to IL-17D can be an antibody, antibody fragment or single-chain antibody that binds to IL-17D, a soluble receptor that binds to IL-17D. Also an antagonist to the IL-17D receptor can be used to treat the disease, such as an antibody, antibody fragment, single-chain antibody or small molecule that binds to the IL-17D receptor. Also an anti-sense nucleotide that binds to the mRNA that encodes IL-17D can be used as an antagonist. A preferred antagonist to IL-17D is the Membrane-Spanning Protein-5 (MSP-5), SEQ ID NOs.: 12 and 13, the mature extracellular portion of the polypeptide being comprised of SEQ ID NO: 14. A preferred embodiment is a soluble receptor SEQ ID NO: 15, corresponding to amino acid residues 879-898 of SEQ ID NO:13, or the soluble receptor SEQ ID NO: 16 corresponding to amino acid residues 856-875 of SEQ ID NO:13.

IL-17D is defined and methods for producing it and antibodies to IL-17D are contained in International Patent Application No. PCT/US99/21677, filed Sep. 17, 1999, and U.S. patent application Ser. No. 09/397,846 filed Sep. 17, 1999. The polynucleotide and polypeptide of human IL-17D are represented by SEQ ID NOs: 1-8, and mouse IL-17D by SEQ ID NOs: 9-11. The MSP-5 sequences are SEQ ID NOs: 12-14, described in International Patent Application No. PCT/US99/05073 and SEQ ID NOs: 15 and 16 are extracellular domains. Another inhibitor would be an anti-idiotypic antibody to MSP-5 that also binds to IL-17D. The present invention also comprises a method for down-regulating IL-17D comprising administering an MSP-5 polypeptide that binds to IL-17D to an individual.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. The binding affinity of an antibody can be readily determined by one of ordinary skill in the art, for example, by Scatchard analysis. A variety of assays known to those skilled in the art can be utilized to detect antibodies that bind to protein or peptide. Exemplary assays are described in detail in Antibodies: A Laboratory Manual, Harlow and Lane (Eds.) (Cold Spring Harbor Laboratory Press, 1988). Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay.

Use of Antagonist to IL-17D to Treat Psoriasis

As indicated in the discussion above and the examples below, IL-17D is involved in the pathology of psoriasis. The present invention is in particular a method for treating psoriasis by administering antagonists to IL-17D. The antagonists to IL-17D can either be a soluble receptor such as SEQ ID NOs: 15 and 16 that binds to IL-17D or antibodies, single chain antibodies or fragments of antibodies that bind to either IL-17D or the IL-17D receptor (SEQ ID NOs: 13 and 14).

Administration of Antagonists to IL-17D

The quantities of antagonists to IL-17D necessary for effective therapy will depend upon many different factors, including means of administration, target site, physiological state of the patient, and other medications administered. Thus, treatment dosages should be titrated to optimize safety and efficacy. Typically, dosages used in vitro may provide useful guidance in the amounts useful for in vivo administration of these reagents. Animal testing of effective doses for treatment of particular disorders will provide further predictive indication of human dosage. Methods for administration include oral, intravenous, peritoneal, intramuscular, transdermal or administration into the lung or trachea in spray form by means or a nebulizer or atomizer. Pharmaceutically acceptable carriers will include water, saline, buffers to name just a few. Dosage ranges would ordinarily be expected from 1 μg to 1000 μg per kilogram of body weight per day. A dosage of MSP-5 or an antibody that binds to IL-17D would be about 25 mg given twice weekly. For subcutaneous or intravenous administration of the antagonist to IL-17D, the antibody or MSP-5 can be in phosphate buffered saline. Also in skin diseases such as psoriasis, the antagonist to IL-17D can be administered via an ointment or transdermal patch. The doses by may be higher or lower as can be determined by a medical doctor with ordinary skill in the art. For a complete discussion of drug formulations and dosage ranges see Remington's Pharmaceutical Sciences, 18^{th} Ed., (Mack Publishing Co., Easton, Pa., 1996), and Goodman and Gilman's: The Pharmacological Bases of Therapeutics, 9th Ed. (Pergamon Press 1996).

The invention is further illustrated by the following non-limiting examples.

### EXAMPLE 1

### In Situ Hybridization of IL-17D

Spatial distribution of IL-17D mRNA in normal and diseased skin tissues were studied using in situ hybridization analysis. In the skin sample analyzed, only psoriasis samples have keratinocyte signal. The hybridization results indicated that IL-17D was highly expressed in the skin of psoriasis patients, and to a lesser degree in the skin of patients with lichen planus.

### EXAMPLE 2

### MSP-5 Binds to IL-17D

Two assays were used to determine that MSP-5 binds to IL-17D. The first was a secretion trap technique, which revealed that MSP-5 expressed from the human keratinocyte cell line, HaCAT bound to IL-17Dβ-9. This was confirmed by transfecting BHK cells with the MSP-5 cDNA and showing that these transfected cells bound to iodinated IL-17Dβ-9.

What is claimed is:

1. A method for treating a mammal afflicted with psoriasis comprising administering an antagonist to a polypeptide selected from the group consisting of SEQ ID NOs: 2, 3, 4, 5, 7, 8, 10 and 11.

2. The method of claim 1 wherein the antagonist is an antibody, antibody fragment or a single-chain antibody.

3. The method of claim 1 wherein the antagonist comprises a polypeptide selected from the group consisting of SEQ ID NOs: 13, 14, 15 and 16 or a subsequence thereof.

4. A method for down-regulating a polypeptide selected from the group consisting of SEQ ID NOs: 2, 3, 4, 5, 7, 8, 10 and 11 comprising administering a therapeutically effective dose of SEQ ID NOs:15 or 16.

5. The use of an antagonist to a polypeptide selected from the group consisting of SEQ ID NOs: 2, 3, 4, 5, 7, 8, 10 and 11 for the production of a medicament for the treatment of psoriasis or psoriatic arthritis.

6. The use of claim 5 wherein the antagonist is an antibody, antibody fragment or single-chain antibody that binds to said polypeptide.

7. The use of claim 5 wherein the antagonist is a polypeptide selected from the group consisting of SEQ ID NOs: 13, 14, 15 and 16 or a subsequence thereof.

8. The use of claim 5 wherein the antagonist is an antibody, antibody fragment or single-chain antibody that binds to a polypeptide selected from the group consisting of SEQ ID NOs: 13, 14, 15 and 16.

9. The method of claim 2 wherein the antibody, antibody fragment, or single chain antibody specifically binds to SEQ ID NO:2.

10. The method of claim 2 wherein the antibody, antibody fragment, or single chain antibody specifically binds to SEQ ID NO: 3.

11. The method of claim 2 wherein the antibody, antibody fragment, or single chain antibody specifically binds to SEQ ID NO:4.

12. The method of claim 2 wherein the antibody, antibody fragment, or single chain antibody specifically binds to SEQ ID NO:5.

13. The method of claim 2 wherein the antibody, antibody fragment, or single chain antibody specifically binds to SEQ ID NO:7.

14. The method of claim 2 wherein the antibody, antibody fragment, or single chain antibody specifically binds to SEQ ID NO:8.

15. The method of claim 2 wherein the antibody, antibody fragment, or single chain antibody specifically binds to SEQ ID NO:10.

16. The method of claim 2 wherein the antibody, antibody fragment, or single chain antibody specifically binds to SEQ ID NO:11.


SEQUENCE LISTING (16 sequences)

SEQ ID NO: 1 - DNA, 1819 bp, Homo sapiens
  CDS (71)...(676)
cgggcgcggggcgcaggcgggctcctccggcgcgtgcggacgctgagcgtggcctgtccctcaggtctggatgctggtagccggcttcctgctggcgctgccgccgagctgggccgcgggcgccccgagggcgggcaggcgccccgcgcggccgcggggctgcgcggaccggccggaggagctactggagcagctgtacgggcgcctggcggccggcgtgctcagtgccttccaccacacgctgcagctggggccgcgtgagcaggcgcgcaacgcgagctgcccggcagggggcaggcccgccgaccgccgcttccggccgcccaccaacctgcgcagcgtgtcgccctgggcctacagaatctcctacgacccggcgaggtaccccaggtacctgcctgaagcctactgcctgtgccggggctgcctgaccgggctgttcggcgaggaggacgtgcgcttccgcagcgcccctgtctacatgcccaccgtcgtcctgcgccgcacccccgcctgcgccggcggccgttccgtctacaccgaggcctacgtcaccatccccgtgggctgcacctgcgtccccgagccggagaaggacgcagacagcatcaactccagcatcgacaaacagggcgccaagctcctgctgggccccaacgacgcgcccgctggcccctgaggccggtcctgccccgggaggtctccccggcccgcatcccgaggcgcccaagctggagccgcctggagggctcggtcggcgacctctgaagagagtgcaccgagcaaaccaagtgccggagcaccagcgccgcctttccatggagactcgtaagcagcttcatctgacacgggaatccctggcttgcttttagctacaagcaagcagcgtggctggaagctgatgggaaacgacccggcacgggcatcctgtgtgcggcccgcatggagggtttggaaaagttcacggaggctccctgaggagcctctcagatcggctgctgcgggtgcagggcgtgactcaccgctgggtgcttgccaaagagatagggacgcatatgctttttaaagcaatctaaaaataataataagtatagcgactatatacctacttttaaaatcaactgttttgaatagaggcagagctattttatattatcaaatgagagctactctgttacatttcttaacatataaacatcgttttttacttcttctggtagaattttttaaagcataattggaatccttggataaattttgtagctggtacactctggcctgggtctctgaattcagcctgtcaccgatggctgactgatgaaatggacacgtctcatctgacccactcttccttccactgaaggtcttcacgggcctccaggtggaccaaagggatgcacaggcggctcgcatgccccagggccagctaagagttccaaagatctcagatttggttttagtcatgaatacataaacagtctcaaactcgcacaattttttcccccttttgaaagccactggggccaatttgtggttaagaggtggtgagataagaagtggaacgtgacatctttgccagttgtcagaagaatccaagcaggtattggcttagttgtaagggctttaggatcaggccgaatatgaggacaaagtgggccacgttagcatctgcagagatcaatctggaggcttctgtttctgcattctgccacgagagctaggtccttgatcttttctttagattgaaagtctgtctctgaacacaattatttgtaaaagttagaagttcttttttaaatcattaaaagaggcttgctga

SEQ ID NO: 2 - protein, 202 aa, Homo sapiens
MLVAGFLLALPPSWAAGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 3 - protein, 187 aa, Homo sapiens
AGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 4 - protein, 186 aa, Homo sapiens
GAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 5 - protein, 185 aa, Homo sapiens
APRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 6 - DNA, 2361 bp, Homo sapiens
  CDS (572)...(1202)
gaattcggcacgagggtcagggaagtattcagtgctttgttgtagagttgttggatagaggcacaggatcatttcatgttgttgaggagaaaggagcaacagcctcctcccaccttattaaaaatagagatttaaaaaaacctctaatttcctcgaagtacagaatctcaagaggtagctctaaggagaatccctctgggtttgagcgcattcctcttccagggggcctattcttggactgctttccttaatagagaaatctctctgagccaaaatcggcctcccccaattccatcctgtcggccccacttttctgctccggagacttccaagccagtccccactcctccttcagccagtcgggcccgcacccgcgcccggcagggccagccctctcctcctcctgcgtggcgcagcacaggccctgagcgcgcgaccccaggccctgggcgccccgccgcatgctcgcggctggaagccccagtttgcgtggcccttcgggttattccgctcaagagccgccgcgtcgccccatctcggcgcgaatctgaaagcgctttcgggggagaagatgttgggggcactggtctggatgctggtagccggcttcctgctggcgctgccgccgagctgggccgcgggcgccccgagggcgggcaggcgccccgcgcggccgcggggctgcgcggaccggccggaggagctactggagcagctgtacgggcgcctggcggccggcgtgctcagtgccttccaccacacgctgcagctggggccgcgtgagcaggcgcgcaacgcgagctgcccggcagggggcaggcccgccgaccgccgcttccggccgcccaccaacctgcgcagcgtgtcgccctgggcctacagaatctcctacgacccggcgaggtaccccaggtacctgcctgaagcctactgcctgtgccggggctgcctgaccgggctgttcggcgaggaggacgtgcgcttccgcagcgcccctgtctacatgcccaccgtcgtcctgcgccgcacccccgcctgcgccggcggccgttccgtctacaccgaggcctacgtcaccatccccgtgggctgcacctgcgtccccgagccggagaaggacgcagacagcatcaactccagcatcgacaaacagggcgccaagctcctgctgggccccaacgacgcgcccgctggcccctgaggccggtcctgccccgggaggtctccccggcccgcatcccgaggcgcccaagctggagccgcctggagggctcggtcggcgacctctgaagagagtgcaccgagcaaaccaagtgccggagcaccagcgccgcctttccatggagactcgtaagcagcttcatctgacacgggaatccctggcttgcttttagctacaagcaagcagcgtggctggaagctgatgggaaacgacccggcacgggcatcctgtgtgcggcccgcatggagggtttggaaaagttcacggaggctccctgaggagcctctcagatcggctgctgcgggtgcagggcgtgactcaccgctgggtgcttgccaaagagatagggacgcatatgctttttaaagcaatctaaaaataataataagtatagcgactatatacctacttttaaaatcaactgttttgaatagaggcagagctattttatattatcaaatgagagctactctgttacatttcttaacatataaacatcgttttttacttcttctggtagaattttttaaagcataattggaatccttggataaattttgtagctggtacactctggcctgggtctctgaattcagcctgtcaccgatggctgactgatgaaatggacacgtctcatctgacccactcttccttccactgaaggtcttcacgggcctccaggtggaccaaagggatgcacaggcggctcgcatgccccagggccagctaagagttccaaagatctcagatttggttttagtcatgaatacataaacagtctcaaactcgcacaattttttcccccttttgaaagccactggggccaatttgtggttaagaggtggtgagataagaagtggaacgtgacatctttgccagttgtcagaagaatccaagcaggtattggcttagttgtaagggctttaggatcaggccgaatatgaggacaaagtgggccacgttagcatctgcagagatcaatctggaggcttctgtttctgcattctgccacgagagctaggtccttgatcttttctttagattgaaagtctgtctctgaacacaattatttgtaaaagttagaagttcttttttaaatcattaaaagaggcttgctgaaaaaaaaaaaaaaaaaaaaa

SEQ ID NO: 7 - protein, 209 aa, Homo sapiens
MLGALVWMLVAGFLLALPPSWAAGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 8 - protein, 187 aa, Homo sapiens; identical to SEQ ID NO: 3

SEQ ID NO: 9 - DNA, 1221 bp, Mus musculis
  CDS (79)...(693)
gggtgtcgcccttatttacttcgcagaagagccttcagcccccctcctaacaagtctggaaagcatcacggcgacgcgatgttggggacactggtctggatgctcgcggtcggcttcctgctggcactggcgccgggccgcgcggcgggcgcgctgaggaccgggaggcgcccggcgcggccgcgggactgcgcggaccggccggaggagctcctggagcagctgtacgggcggctggcggccggcgtgctcagcgccttccaccacacgctgcagctcgggccgcgcgagcaggcgcgcaatgccagctgcccggccgggggcagggccgccgaccgccgcttccggccacccaccaacctgcgcagcgtgtcgccctgggcgtacaggatttcctacgaccctgctcgctttccgaggtacctgcccgaagcctactgcctgtgccgaggctgcctgaccgggctctacggggaggaggacttccgctttcgcagcacacccgtcttctctccagccgtggtgctgcggcgcacagcggcctgcgcgggcggccgctctgtgtacgccgaacactacatcaccatcccggtgggctgcacctgcgtgcccgagccggacaagtccgcggacagtgcgaactccagcatggacaagctgctgctggggcccgccgacaggcctgcggggcgctgatgccggggactgcccgccatggcccagcttcctgcatgcatcaggtcccctggccctgacaaaacccaccccatgatccctggccgctgcctaatttttccaaaaggacagctacataagctttaaatatatttttcaaagtagacactacatatctacaactattttgaatagtggcagaaactattttcatattagtaatttagagcaagcatgttgtttttaaacttctttgatatacaagcacatcacacacatcccgttttcctctagtaggattcttgagtgcataattgtagtgctcagatgaacttccttctgctgcactgtgccctgtccctgagtctctcctgtggcccaagcttactaaggtgataatgagtgctccggatctgggcacctaaggtctccaggtccctggagagggagggatgtgggggggctagaaccaagcgcccctttgttctttagcttatggatggtcttaactttataaagattaaagtttttggtgttattctttca

SEQ ID NO: 10 - protein, 205 aa, Mus musculis
MLGTLVWMLAVGFLLALAPGRAAGALRTGRRPARPRDCADRPEELLEQLYGRLAAGVLSAFHHTLQLGPREQARNASCPAGGRAADRRFRPPTNLRSVSPWAYRISYDPARFPRYLPEAYCLCRGCLTGLYGEEDFRFRSTPVFSPAVVLRRTAACAGGRSVYAEHYITIPVGCTCVPEPDKSADSANSSMDKLLLGPADRPAGR

SEQ ID NO: 11 - protein, 183 aa, Mus musculis
AGALRTGRRPARPRDCADRPEELLEQLYGRLAAGVLSAFHHTLQLGPREQARNASCPAGGRAADRRFRPPTNLRSVSPWAYRISYDPARFPRYLPEAYCLCRGCLTGLYGEEDFRFRSTPVFSPAVVLRRTAACAGGRSVYAEHYITIPVGCTCVPEPDKSADSANSSMDKLLLGPADRPAGR

SEQ ID NO: 12 - DNA, 3016 bp, Homo sapiens
  CDS (12)...(2864)
tggattacacaatgacatggagaatgggaccccgtttcactatgctgttggccatgtggctagtgtgtggatcagaaccccacccccatgccactattagaggcagccacggaggacggaaagtgcctttggtttctccggacagcagtaggccagctcggtttctgaggcacactgggaggtctcgcggaattgagagatccactctggaggaaccaaaccttcagcctctccagagaaggaggagtgtgcccgtgttgagactagctcgcccaacagagccgccagcccgctcggacatcaatggggccgccgtgagacctgagcaaagaccagcagccaggggctctccgcgtgagatgatcagagatgaggggtcctcagctcggtcaagaatgttgcgtttcccttcggggtccagctctcccaacatccttgccagctttgcagggaagaacagagtatgggtcatctcagcccctcatgcctcggaaggctactaccgcctcatgatgagcctgctgaaggacgatgtgtactgtgagctggcggagaggcacatccaacagattgtgctcttccaccaggcaggtgaggaaggaggcaaggtgagaaggatcaccagcgagggccagatcctggagcagcccctggaccctagcctcatccctaagctgatgagcttcctgaagctggagaagggcaagtttggcatggtgctgctgaagaagacgctgcaggtggaggagcgctatccatatcccgttaggctggaagccatgtacgaggtcatcgaccaaggccccatccgtaggatcgagaagatcaggcagaagggctttgtccagaaatgtaaggcctctggtgtagagggccaggtggtggcggaggggaatgacggtggagggggagcaggaaggccaagcctgggcagcgagaagaagaaagaggacccaaggagagcacaagtcccaccaaccagagagagtcgggtgaaggtcctgagaaaactggccgccactgcaccagctttgccccaacctccctcaacccccagagccaccacccttcctcctgccccagccacaacagtgactcggtccacgtcccgggcggtaacagttgctgcaagacctatgaccaccactgcctttcccaccacgcagaggccctggaccccctcaccctcccacaggccccctacaaccactgaggtgatcactgccaggagaccctcagtttcagagaatctttaccctccatcccggaaggatcagcacagggagaggccacagacaaccaggaggcccagcaaggccaccagcttggagagcttcacaaatgcccctcccaccaccatctcagaacccagcacaagggctgctggcccaggccgtttccgggacaaccgcatggacaggcgggaacatggccaccgagacccaaatgtggtgccaggtcctcccaagccagcaaaggagaaacctcccaaaaagaaggcccaggacaaaattcttagtaatgagtatgaggagaagtatgacctcagccggcctactgcctctcagctggaggacgagctgcaggtggggaatgttccccttaaaaaagcaaaggagtctaaaaagcatgaaaagcttgagaaaccagagaaggagaagaaaaaaaagatgaagaatgagaacgcagacaagttacttaagagtgaaaagcaaatgaagaagtctgagaaaaagagcaagcaagagaaagagaagagcaagaagaaaaaaggaggtaaaacagaacaggatggctatcagaaacccaccaacaaacacttcacgcagagtcccaagaagtcagtggccgacctgctggggtcctttgaaggcaaacgaagactccttctgatcactgctcccaaggctgagaacaatatgtatgtgcaacaacgtgatgaatatctggaaagtttctgcaagatggctaccaggaaaatctctgtgatcaccatcttcggccctgtcaacaacagcaccatgaaaatcgaccactttcagctagataatgagaagcccatgcgagtggtggatgatgaagacttggtagaccagcgtctcatcagcgagctgaggaaagagtacggaatgacctacaatgacttcttcatggtgctaacagatgtggatctgagagtcaagcaatactatgaggtaccaataacaatgaagtctgtgtttgatctgatcgatactttccagtcccgaatcaaagatatggagaagcagaagaaggagggcattgtttgcaaagaggacaaaaagcagtccctggagaacttcctatccaggttccggtggaggaggaggttgctggtgatctctgctcctaacgatgaagactgggcctattcacagcagctctctgccctcagtggtcaggcgtgcaattttggtctgcgccacataaccattctgaagcttttaggcgttggagaggaagttgggggagtgttagaactgttcccaattaatgggagctctgttgttgagcgagaagacgtaccagcccatttggtgaaagacattcgtaactattttcaagtgagcccggagtacttctccatgcttctagtcggaaaagacggaaatgtcaaatcctggtatccttccccaatgtggtccatggtgattgtgtacgatttaattgattcgatgcaacttcggagacaggaaatggcgattcagcagtcactggggatgcgctgcccagaagatgagtatgcaggctatggttaccatagttaccaccaaggataccaggatggttaccaggatgactaccgtcatcatgagagttatcaccatggatacccttactgagcagaaatatgtaaccttagactcagccagtttcctctgcagctgctaaaactacatgtggccagctccattcttccacactgcgtactacatttcctgcctttttctttcagtgtttttctaagactaaataaatagcaaactttcaccta

SEQ ID NO: 13 - protein, 950 aa, Homo sapiens
MTWRMGPRFTMLLAMWLVCGSEPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 14 - protein, 929 aa, Homo sapiens
EPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 15 - protein, 19 aa, Homo sapiens
YPSPMWSMVIVYDLIDSMQ

SEQ ID NO: 16 - protein, 20 aa, Homo sapiens
YFQVSPEYFSMLLVGKDGNV